(19) United States
(12) Patent Application Publication
(10) Pub. No.: US 2021/0346558 A1
(43) Pub. Date: Nov. 11, 2021

(54) ROBOTIC GERMICIDAL IRRADIATION SYSTEM

(71) Applicant: Chenghung Pan, Palo Alto, CA (US)

(72) Inventor: Chenghung Pan, Palo Alto, CA (US)

(21) Appl. No.: 17/246,524

(22) Filed: Apr. 30, 2021

Related U.S. Application Data
(60) Provisional application No. 63/022,488, filed on May 9, 2020.
(60) Provisional application No. 63/033,209, filed on Jun. 2, 2020.
(60) Provisional application No. 63/111,317, filed on Nov. 9, 2020.

(51) Int. Cl. A61L 2/24; A61L 2/28; A61L 2/10; G05D 1/02

(52) U.S. Cl. CPC A61L 2/24; A61L 2/28; A61L 2/10; G05D 1/0278; A61L 2202/16; A61L 2202/25; A61L 2202/11; A61L 2202/14; G05D 1/0219

(57) ABSTRACT

A smart ultraviolet germicidal irradiation (UVGI) system automatically tracks irradiation times and irradiation locations within an environment being disinfected. The UVGI system can employ or execute a pre-determined disinfection plan that provides a time schedule and geographic routes for one or more mobile UV sources. For example, a replaceable dolly or robot of the UVGI system may be programmed to navigate a space based on the pre-determined disinfection plan.

### CROSS-REFERENCE TO RELATED APPLICATIONS

This patent document is claims benefit of the earlier filing date of U.S. provisional Pat. App. No. 63/022,488, entitled “An Ultraviolet Germicidal Irradiation Room Analysis Method,” filed May 9, 2020, U.S. provisional Pat. App. No. 63/033,209, entitled “A Smart Ultraviolet Germicidal Irradiation System,” filed Jun. 2, 2020, and U.S. provisional Pat. App. No. 63/111,317, entitled “A UVC Robotic Platform,” filed Nov. 9, 2020, all of which are hereby incorporated by reference in their entirety.

### BACKGROUND

Ultraviolet (UV) light is electromagnetic radiation with wavelengths shorter than visible light, i.e., shorter than about 400 nm, but longer than X-rays, i.e., longer than about 10 nm. UV light may be categorized by wavelength range, with short-wavelength UV (UVC) having wavelengths shorter than about 300 nm being considered “germicidal UV” because nucleic acids in microorganisms such as bacteria or viruses strongly absorb UV wavelengths between about 200 nm and 300 nm and the absorbed energy from the UV radiation can result in the death or deactivation of the microorganisms.

People generally need to be protected from UV light exposure during UV disinfection processes. For example, during UV disinfection of a room, people may be required to leave the room before activation of a UV source, and people may need to be kept out of the room until the UV source is shut off. As a result, operation of conventional UV disinfectors to disinfect an extended area may be a cumbersome process requiring an operator to place a UV disinfector in the proper location for disinfection of part of an extended area, leave the area of the UV disinfector for the time the UV disinfector is active, then return to move the UV disinfector and repeat the process as many times as needed to cover the entire extended area. Alternatively, many UV disinfectors may be purchased for simultaneous activation, but the number of UV disinfectors required increases solution costs.

### BRIEF DESCRIPTION OF THE DRAWINGS

FIG. 1 shows a plan view of a facility and a route of a robotic UV disinfector in accordance with an example of the present disclosure.

FIG. 2 shows a smart ultraviolet germicidal irradiation system in accordance with an example of the present disclosure providing control systems to automatically track and control operation of a robotic UV disinfector.

FIG. 3 shows a robotic UV disinfector in accordance with an example of the present disclosure.

FIG. 4 is a block diagram of a motorized dolly in an ultraviolet germicidal irradiation system in accordance with an example of the present disclosure.

FIGS. 5-1 and 5-2 show a disinfection system including a universal robotic dolly and a separable or standalone multifunction UV disinfector in accordance with an example of the present disclosure.

FIG. 6 is a flow diagram of a training process for a UV disinfection system in accordance with an example of the present disclosure.

FIG. 7 is a flow diagram of a disinfection process in accordance with an example of the present disclosure.

The drawings illustrate examples for the purpose of explanation and are not of the invention itself. Use of the same reference symbols in different figures indicates similar or identical items.

### DETAILED DESCRIPTION

A smart UltraViolet Germicidal Irradiation (UVGI) system automatically performs UV disinfection processes for extended areas, e.g., one or more rooms in a facility. The UVGI system may include a mobile robot that is programmed or controlled to move to multiple locations, which may be identified in a predetermined disinfection plan. The disinfection plan may, for example, include data that defines a schedule, routes or locations, and operations to be perform while the robot is moving or at the locations. Disinfection plans may be programmed into or learned by the UVGI system. For example, an operator may direct a UVGI robot on a route for a disinfection process, and a UVGI system may track and record movements or locations. The operator may program, enter, or select exposure times during learning of the disinfection process, and the UVGI system may later automatically replay the disinfection process by moving to the recorded locations at which the UVGI system emits UV light for recorded durations.

In one example implementation, a UVGI system includes a mobile robot with an integrated UV source that the robot activates upon reaching target locations. In another implementation, the UVGI system employs a programmable dolly capable of following or retracing a route to a set of target locations for UV irradiation. The dolly may be configured to carry any desired disinfection system, e.g., a standalone germicidal system including a source of germicidal UV light, to a sequence of locations. The dolly or the germicidal system may employ a mobile power source and may include control logic or may communicate with control logic of the UVGI system that activates and deactivates UV irradiation according to a disinfection plan.

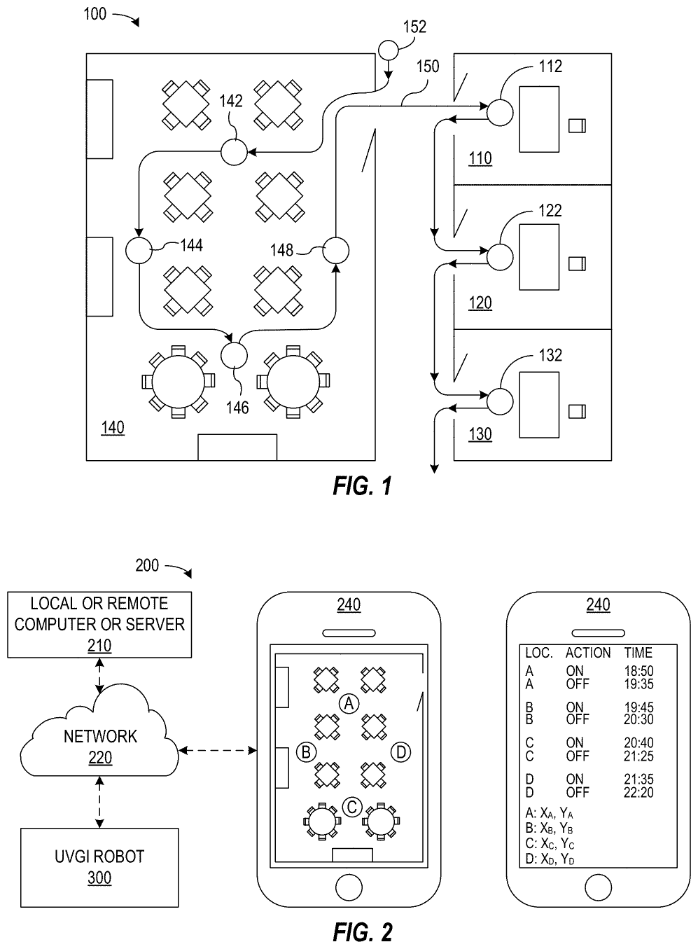

FIG. 1 shows a floorplan of an area or environment 100 to be disinfected using a UVGI system in accordance with an example of the present disclosure. Area 100 includes multiple rooms 110, 120, 130, and 140, each of which includes ceilings, walls, floors, and furnishings having surfaces that might harbor microorganisms that need to be deactivated. Some rooms, e.g., rooms 110, 120, and 130, may have a size and shape that permits efficient disinfection from using a suitable UVGI system at a single location within the room, e.g., location 112, 122, or 132 in room 110, 120, or 130. Other rooms, e.g., room 110, may be relatively large or irregularly shaped or furnished so that effective disinfection requires positioning and operating a germicidal UV source at multiple locations, e.g., 112, 114, 116, and 118 in room 140. In general, determining suitable or optimal locations for operation of a UVGI system may involve using general rules and testing, e.g., trial and error and may be a bias toward over irradiating areas. Alternatively, U.S. patent application Ser. No. 17/092,010, entitled “Ultraviolet Germicidal Irradiation Room Analysis,” filed Nov. 6, 2020, which is hereby incorporated by reference in its entirety, describes systems and methods for identifying a plan for efficiently and effectively disinfecting a room using a UVGI system.

The floorplan of FIG. 1 shows spots or target locations 112, 122, 132, 142, 144, 146, and 148 where a UVGI equipment should be placed and operated to disinfect rooms 110, 120, 130, and 140. For each location, the UVGI equipment needs to expose the surrounding local area to germicidal UV radiation for a time or duration that is sufficient to effectively deactivate target microorganisms in the local area. Above-incorporated U.S. patent application Ser. No. 17/092,010 describes methods and processes for efficient locations and durations for target microorganisms, but other techniques such as following geometric rules for positioning, exposure times expected to be long than necessary, along with testing may be used to identify a plan including locations and exposure durations associated with the locations. Depending on the capabilities of the UVGI equipment, operation at a location may simply involve emitting germicidal UV radiation in all or most directions around the location 112, 122, 132, 142, 144, 146, or 148 or may involve a routine that moves a UV source or directs UV light from the UVGI equipment in programmed directions.

FIG. 1 further shows a route 150 that UVGI equipment can traverse to reach locations 142, 144, 146, 148, 112, 122, and 132 during a disinfection process. In some situations, the UVGI equipment may keep a UV source active while moving along route 150. As described further below, UVGI equipment may generally include a robot or self-navigating dolly that is able to automatically traverse route 150 and pause at target locations 142, 144, 146, 148, 112, 122, and 132 during a disinfection process.

FIG. 2 illustrates a UVGI system 200 in accordance with another example of the present disclosure. As shown in FIG. 2, UVGI system 200 includes a computer system 210 connected through a network 220 to a UVGI robot 300 and to a client or user device 240 (such as a PC, a tablet computer or a smartphone). Computer system 210 may be a remote cloud server and network 220 may include one or more public network, e.g., the Internet, and one or more local area network, e.g., a wireless network that covers locations in the extended area to be disinfected. Alternatively, computer system 210 may employ a local computer, e.g., a Personal Computer (PC), laptop computer, tablet computer, or other computing device that is on a local private network and is able to maintain a database containing a disinfection plan data for the area to be disinfected. The disinfection plan data may particularly identify all switch on/off timing and location information for a disinfection process. Computer system 210 may further program, control, or operate UVGI robot 300 to perform the disinfection process and/or may enable a user of client device 240 to display the tracking information of the disinfection process and initiate, control, or alter a disinfection process or a plan for a subsequent disinfection process.

FIG. 2 particularly illustrates a user interface on client device 240 displaying a floorplan of an area disinfected and identifying locations where UVGI robot 300 is scheduled to stop. The floorplan may be overlaid with color coding that indicates the UV dose at each point in the area disinfected. A second display on client device 240 may provide a text display indicating a series actions during the disinfection process and for each action indicating: what action is taken, e.g., turning a UV source on or off; a location of UVGI robot 300 when the action is taken; and a time when the action is taken. The locations for the actions may be given using local coordinates, e.g., X and Y displacements from a local reference point, or global components, e.g., Global Positioning System (GPS) coordinates. The user interface on device 240 may permit a user to communicate with server 210 or UVGI robot 300 to initiate, alter, or abort a disinfection process.

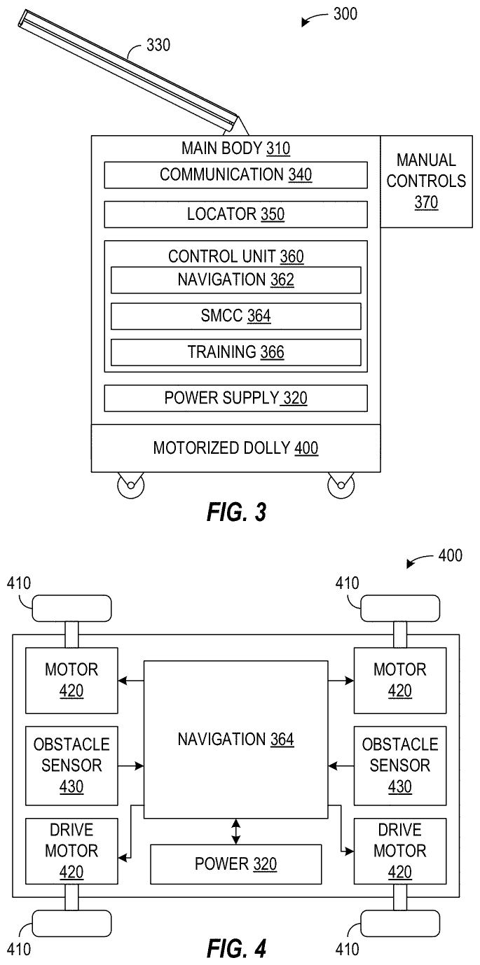

FIG. 3 shows an example of a UVGI robot 300 in accordance with an example of the present disclosure. UVGI robot 300 includes: a main body 310; a power supply 320; a set of UVC lamps 330; a wireless communication device 340 such as an internet modem; a locator module 350 such as a GPS module; a control module 360, manual controls 370, and a motorized dolly 400. Power supply 320 includes batteries or other mobile power sources and may provide electrical power to all systems and modules on UVGI robot 300 including UV lamps 330, communication module 340, locator module 350, control module 360, and motorized dolly 400. UVGI robot 300 uses communication module 340 to communicate through a wireless network, e.g., to receive commands or disinfection plan information from a local or cloud-based server or other computer system. Manual controls 370, which may include buttons, switches, a touchscreen, a Bluetooth interface to nearby control devices, or any other user interface devices, allow an operator to manually control UVGI robot 300. For example, an operator may use manual controls 370 to power up or shut down robot 300, activate available operating modes, e.g., a training mode or disinfection mode, of UVGI robot 300, or steer robot 300. Control module 360 processes the commands or disinfection plan information, receives location information or coordinates from locator module 350, controls motorized dolly 400 to move robot 300 to target locations, and activates and operates UV lamps 330 to disinfect surfaces in the environment around the target locations.

Control module 360 may include a microcontroller with interfaces for control of other modules of UVGI robot 300 and suitable software or firmware to control navigation of robot 300 and training or operation of disinfection functions. As shown, two primary modules implemented in control unit 360 include a navigation module 362, a Switching Control and Monitor Module (SCMM) 364, and a training module 366. The functions of navigation module 362 may include receiving or learning and storing a route or target locations for a disinfection process and controlling motorized dolly 400 to move robot system 330 during performance of a disinfection process. As described further below, navigation unit 362 may operate autonomously to detect and avoid obstacles while navigating a route for a disinfection process. SCMM 364 controls disinfection operations such as activation of a UV lamp at a target location for a duration sufficient to disinfect surfaces in the environment around the target location. SCMM 364 may further monitor performance of disinfection systems and may monitor the environment for safety. In particular, SCMM 354 may employ motion or occupancy sensors to detect whether people are present in the environment around robot 300 and deactivate disinfection functions upon detection of safety concerns. Training module 366 may be used to record disinfection plan data such as steering commands that navigate UVGI robot from one location to the next in a disinfection plan, the measured coordinates of target locations, and timing for disinfection operations to be performed at the target locations.

Operation of UVGI robot 300 may be initiated when UVGI robot 300 is activated near or within an area to be disinfected. For example, in area 100 shown by the floorplan of FIG. 1, UVGI robot 300 may be at a starting location 152 of route 150 when activated and placed in a disinfection mode. Control unit 360 is turned on and receives though communication device 340 or already has disinfection plan information. For example, UVGI robot 300 may have been previously programmed with (or receive from computer system 210 of FIG. 2) the coordinates of target locations, information identifying a route to traverse, or timing information indicating when to turn-on/turn-off UV lamps 330 or how long to operate a disinfection system at each target location or along the route. Locator module 350 reports the current location coordinates of UVGI robot 300 to control module 360 or to the remote cloud server 210 through communication module 340. Control module 360 (specifically navigation module 362) can then operate motorized dolly 400 to move UVGI robot 300 to a first target location (e.g., location 142 in FIG. 1) in the disinfection plan, and then if the target area is safe, SCMM 365 activates UV lamps 330 for a duration or time interval indicated in the disinfection plan. UVGI robot 300 can sequentially move to each of the remaining target locations and complete a disinfection process that the disinfection plan represents. During the disinfection process, UVGI 300 can report each step to server 210, and a database in server 210 can record and document disinfection.

FIG. 4 shows a block diagram of motorized dolly 400 in accordance with one example of the present disclosure. Motorized dolly 400 employs one or more wheel-controlling motors 420 and one or more obstacle detection sensors 430. Navigation module 364 of control unit 360 particularly uses obstacle sensors 420 to detect obstacles in the path of dolly 400 and controls motors 420 to navigate around obstacles (when possible) and move to the next target location. Navigation module 364 can navigate based on a stored route or path transferred from a remote cloud server or local computer, e.g., computer system 210 of FIG. 2 to the disinfection robot. The process for control of drive motors 420 to follow a specified route may be altered or updated based on the information collected from the obstacle detection sensors 430. For example, if dolly 400 is moving along a specified path when front obstacle detection sensors 430 detected an obstacle, control unit 360 may halt dolly 400 and then navigate dolly 400 backward before attempting to plot a path around the obstacle or reporting the obstacle to a local or remote control system, e.g., computer system 210 or user device 240. Another example is if a right front obstacle sensor 430 detects an obstacle, and a left front obstacle sensor 430 does not detect obstacles, control unit 360 may halt dolly 400 and commanded drive motors 420 as needed to make a left turn, e.g., by 10 degrees, to avoid a collision with the detected obstacles and then turn right to return to the stored path or to proceed more directly toward the next target location.

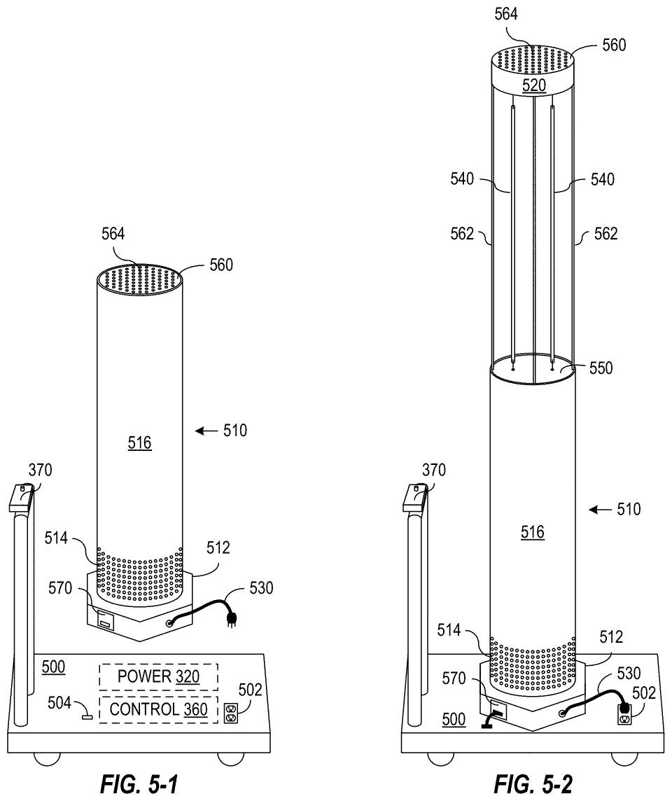

Disinfection systems in accordance with some examples of the present disclosure can employ a universal robotic dooly that is able to work with a variety of disinfection devices. FIG. 5-1, for example, illustrates a disinfection system in accordance with an example of the present disclosure including a universal robotic dolly 500 and a multi-function disinfector 510. Multi-function disinfector 510 may be a standalone device capable of performing disinfection procedures without need of robotic dolly 520. Robotic dolly 520 can carry and control disinfector 510 during a disinfection process.

Universal dolly 500 may include the same components systems as robot 300 of FIG. 3 including motorized dolly 400 of FIG. 4, except that universal dolly 500 does not require UV lamps or other disinfection components that may be provided on the carried disinfector 510. More particularly, universal dolly 500 generally includes a main body or structure 310, a power supply 320, a communication module 340, a locator module 350, a control module 360, manual controls 370, and a motorized dolly 400 having wheels 410, motors 420, and obstacle sensors 430 as described above with reference to FIGS. 3 and 4. Universal dolly 500 does not require UV lamps 330 because disinfector 510 includes disinfection hardware.

FIG. 5-2 shows the disinfection system of FIG. 5-1 in a configuration in which disinfector 510 is sitting or mounted on universal dolly 520. FIG. 5-2 also illustrates that universal dolly 500 may have an outlet 502 such as a standard AC power outlet or a port 504 such as a USB port through which universal dolly 520 can provide power to or communicate with disinfector 510. More generally, universal dolly 520 may not need to provide electrical power or communications to disinfector 510 if disinfector 510 has its own portable power supply or wireless communication module.

Disinfector 510 in the illustrated configuration is a multi-function UV disinfector that is operable for surface disinfection or air disinfection and filtering as described in U.S. patent application Ser. No. 17/138,332, entitled “Multifunction UV Disinfector,” filed Dec. 30, 2020, which is hereby incorporated by reference in its entirety. In this example, disinfector 510 has a sliding mount for UV sources or lamps 540 that can be moved into a cylindrical shielding 516 to reach a closed or shielded configuration as shown in FIG. 5-1 for air disinfection and moved out of cylindrical shield 516 to reach an open or unshielded configuration for surface disinfection as shown in FIG. 5-2. Multifunction disinfector 510 otherwise has a housing including a base 512 and shield 516 attached to base 512. A lower vent 514 is integrated into base 512 or shield 516. Shield 516 is hollow and contains a sliding insert 560, e.g., an insert on a sliding mount and connected to a motor or other drive system. Sliding insert 560 provides a framework 562 on which functional elements, e.g., a blower 520, UV light sources 540, and a filter 550, or UV disinfector 500 may be mounted as shown in FIG. 5-2. Sliding insert 560 also includes an upper vent 564 used during an air disinfecting mode of multifunction UV disinfector 510.

In the closed configuration shown in FIG. 5-1, insert 550 is surrounded by shield 516, and vent 564 is at or near the top of UV disinfector 510. For air disinfection, blower 520 can be activated to push an air flow, e.g., into vent 564, down through shield 516, through filter 550, and out of vent 514. During the air disinfection, UV light sources 540 may be on so that UV light can inactivate contagions in the air flow and that may be trapped in filter 550 or elsewhere inside UV disinfector 510. Shield 516, filter 550, and blower 520 enclose UV light sources 540 so that UV light from UV sources 540 cannot escape from UV disinfector 500 in the closed configuration of FIG. 5-1.

In the open configuration shown in FIG. 5-2, insert 560 is slid up and held in a position where UV sources 540 are exposed and able to emit UV light for surface disinfection in the area around disinfector 510. UV sources 540 may be activated to emit UV light for surface disinfection only when an occupancy detector determines that the surrounding around UV disinfector 500 is unoccupied. An advantage of using an insert 560 able to move UV sources 540 up for surface disinfection is that even if UV disinfector 510 has a low profile, e.g., around 4 feet tall or less, in the closed configuration, the open configuration is taller, e.g., about 6 feet tall, and positions UV light source 540 higher for surface disinfection at a height typical of human activity in a room or other environment to be disinfected.

Multifunction UV disinfector 510 further includes a control system 570 that enables a user interface for control of UV disinfector 500. Control system 470 may provide for wireless or wired communication with a control system such as control module 360 of universal dolly 500 as shown in FIGS. 5-1 and 5-2 or computer 210 or user device 240 of FIG. 2. FIGS. 5-1 and 5-2 show a disinfection system using a wired connection (e.g., USB connection) for signals from control module 360 to control operation of disinfector 510. Alternatively, wireless communications could be provided from disinfector 510 to universal dolly 500 or to a remote system such as computer system 210 or user device 240. Commands to disinfector 510 may thus be sent directly to disinfector 510 or relayed through the communications system available in universal dolly 500.

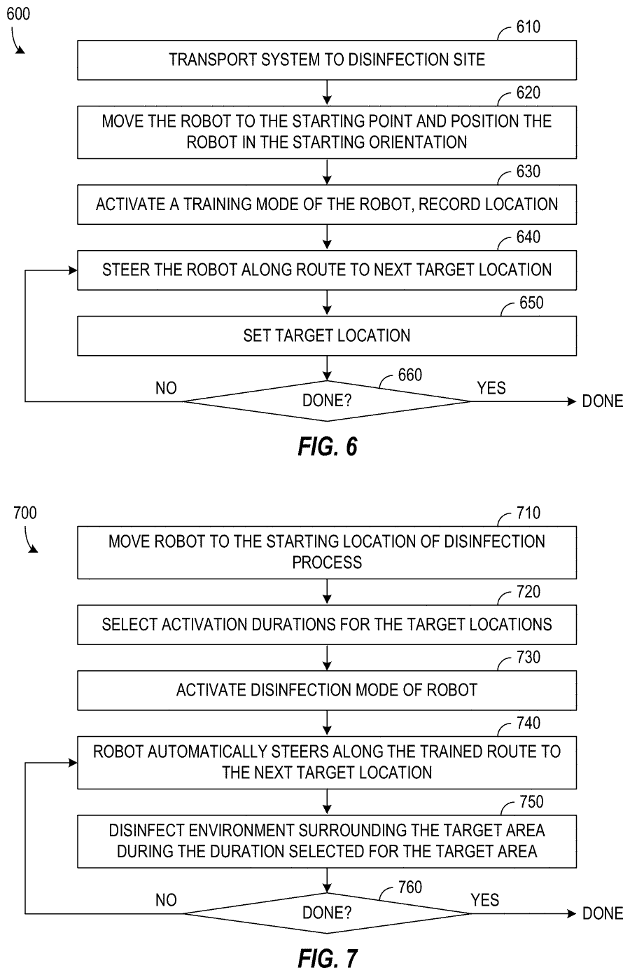

FIG. 6 is a flow diagram of a process 600 for training a UV disinfection system to perform a site disinfection. In an initial process block 610 of training process 600, a UV disinfection robot or a system including a disinfector on a universal dolly is brought to the site to be disinfected where, in a process block 620, a user moves the robot to a starting location. For example, the robot may be positioned at a starting location 152 of route 150 at site 100 of FIG. 1. In process block 630, a user activates a training mode of the robot, and the UV disinfection system marks the starting point, e.g., determines and records the position coordinates that the locator module of the robot measures for the starting location.

In a process block 640, the user instructs the robot, e.g., using manual controls, to move and steers the robot along the desired route to the next target location. In one specific implementation, the user uses a joystick or other control device to send commands to the robot, causing the drive motors operate. The control module of the robot stores or otherwise records the commands that caused the robot to move from its starting location to the next target location, e.g., from location 152 to location 142 in FIG. 1. The robot may relay the commands to the local or remote control computer, e.g., computer system 210 of FIG. 2, and may later replay the commands to follow the same route.

A process block 650 sets or records in the disinfection plan data operating parameters for the disinfection operation at the target. In particular, when the robot reaches the next target location, the operator may use the joystick or other control device to indicate to the robot that the robot is at the next target location. The robot can then record the location, e.g., store a reading of location coordinates from the location module of the robot. A user may also set a type of disinfection operation, a duration, or start and stop times for a disinfection operation at the target location. Alternatively, a duration needed for disinfection of the environment surrounding the target location may be automatically determined or calculated using techniques such as described in above-incorporated U.S. patent application Ser. No. 17/092,010. The duration or on/off times for disinfection may be determined for each target location independently or determine based on all or multiple target locations that may have overlapping disinfection areas.

A decision block 660, after process block 650, determines whether all target locations have been set. For example, the operator may end training mode operation of the robot to indicate the last target location has been set. If the system needs to learn additional target locations for disinfection operations, process 600 returns to process bloc 640 where the operator manually steers the robot to the next target location. Process blocks 640 and 650 may thus be repeated multiple times decision block 660 determines process 600 is done.

FIG. 7 is a flow diagram of a process 700 for performing a disinfection process that may have been trained using learning process 600 of FIG. 6 or other techniques. An initial process block 710 of process 700 moves the disinfection robot to the starting location for disinfection process 700. For example, a user may position and orient the robot at the same starting location used during training. A process block 720 may set durations or start and stop times for disinfection operation to be activated at the target locations of the disinfection process. The durations (or start and stop times) may be based on previously learned or determined durations, the current time, and any further user settings or preferences.

In a process block 730, the user activates a disinfection mode of the robot, e.g., using a switch or button on the robot or using a user device, e.g., user device 240 of FIG. 1. The UV disinfection system upon activation may determine a current location of the robot, e.g., using a locator module on the robot, and compare the current coordinates to coordinates for the starting location and the first target location. The UV disinfection system may then determine if the robot can proceed to the first target location or if the robot needs to reposition or reorient itself to follow the recorded movement commands or otherwise navigate to the first location.

The robot in block 740 autonomously steers itself to the next target location. As mentioned above, the robot has obstacle sensors that are used during movement and steering to detect obstacles that may now be along the desired route. When one or more obstacles are encountered, the robot may determine the locations of the obstacles and may select a maneuver that will bypass the obstacles return the robot to the recorded rout or direct the robot to the next target location. If the robot is unable to determine a safe path forward, the robot may report an error condition requiring user attention.

The robot may confirm reaching the next target location by checking the current location coordinates of the robot to stored coordinates for the target location. Once the robot has confirmed reaching the target location, the robot in a process block 750 may perform a disinfection operation indicated by the disinfection plan. For example, the robot may employ occupancy sensors to confirm that target location is unoccupied. Once safety is confirmed, the robot may activate a UV lamp at a start time the disinfection plan gives for disinfection of the environment around the target location and subsequently deactivate the UV lamp at a stop time the disinfection plan gives for disinfection of the environment around the target location. Alternatively, the robot in process block 750 may activate a UV lamp upon reaching the target location and deactivate the UV lamp after expiration of a duration given in the disinfection plan.

A decision block 760, after process block 750, determines whether all target locations have been disinfected. If the disinfection process needs to disinfect additional target locations, process 700 returns to process bloc 740 where the robot steers itself to the next target location. Process blocks 740 and 750 may thus be repeated multiple times until decision block 760 determines disinfection process 700 is complete.

A UVC robot system as described above may include a Wi-Fi gateway to connect to a cloud-based service. When the UVC robot is connected to the Internet, the robot can track the route of auto-disinfection and store the route and provide dosage map information to the cloud-based service. Users can thus log into an account with the service to review the route tracking and dosage map for every disinfection process a robot has accomplished.

Each of modules disclosed herein may include, for example, hardware devices including electronic circuitry for implementing the functionality described herein. In addition or as an alternative, each module may be partly or fully implemented by a processor executing instructions encoded on a machine-readable storage medium.

All or portions of some of the above-described systems and methods can be implemented in a computer-readable media, e.g., a non-transient media, such as an optical or magnetic disk, a memory card, or other solid state storage containing instructions that a computing device can execute to perform specific processes that are described herein. Such media may further be or be contained in a server or other device connected to a network such as the Internet that provides for the downloading of data and executable instructions.

Although implementations have been disclosed, these implementations are only examples and should not be taken as limitations. Various adaptations and combinations of features of the implementations disclosed are within the scope of the following claims.

What is claimed is:

1. An ultraviolet germicidal irradiation (UVGI) system including a robot, the robot comprising:
a motorized dolly;
a portable power supply on the motorized dolly; and
a control unit coupled to control the motorized dolly and the portable power supply, the control unit providing:
a navigation module that controls the motorized dolly to steer to a target location identified in a disinfection plan data; and
a switching control and monitor module connected to activate a UV disinfector at the target location for a time identified in the disinfection plan data.

2. The system of claim 1, wherein:
the robot further comprises a wireless adapter connected to the control unit; and
the UVGI system further comprises a computer system communicating with the control unit of the robot through the wireless adapter, wherein the computer system comprises one of a computer on a private network with the robot and a remote server that accesses the robot through the Internet.

3. The system of claim 2, wherein the computer system is configured to collect tracking information from the robot and enable a user device to display the tracking information.

4. The system of claim 2, wherein the computer system is configured to collect the disinfection plan data and enable a client to display the disinfection plan data indicating turn on/off timing of activation of the UV disinfector in a mapping format or a tabular format.

5. The system of claim 4, wherein the computer system is configured to enable a client to display a UV dosage map to show UV dose values at each spot in a room to be disinfected.

6. The system of claim 2, where the navigation steers motorized dolly according to a series of control codes that the computer system provides to the robot.

7. The system of claim 1, where the navigation steers motorized dolly according to a series of control codes stored inside the control unit.

8. The system of claim 1, wherein the robot further comprises a Global Position System (GPS) locator configured to determine a location of the robot.

9. The system of claim 1, wherein the robot further comprises the UV disinfector.

10. The system of claim 1, wherein the UVGI system further comprises the UV disinfector as a standalone device operable separate from the robot, the robot being configured to carry the UV disinfector.

11. The system of claim 10, wherein the robot comprises an outlet through which robot connects and provides power to the standalone device while the robot carries the standalone device.

12. The system of claim 10, wherein the robot comprises a port through which robot connects and provides control signals to the standalone device while the robot carries the standalone device.

13. The system of claim 1, wherein the motorized dolly comprises:
a plurality of wheels;
a plurality of motors connected to rotate the wheels; and
a plurality of obstacle detection sensors, wherein
the navigation module is configured to detect output of the obstacle detection sensors and control the motors.

14. The system of claim 1, where the robot further comprises manual controls.

15. The system of claim 14, wherein in the manual controls are operable for:
manual steering the robot to a location;
recording the location of the robot; and
activating a training mode of the robot, wherein in training mode, the recording of the location of the robot sets a target location in the disinfection plan data.

16. The system of claim 15, wherein in the manual controls are further operable for setting of a time for a disinfection operation at the location of the robot, the time being recorded in the disinfection plan data when the training mode is active.

17. The system of claim 15, wherein commands for the manual steering of the robot are recorded in the disinfection plan data during operation of the robot in training mode.

18. The system of claim 15, wherein while training mode is activated, a computer system connected to the robot through a network collects location and time data from the robot in the disinfection plan data.

19. A disinfection method, comprising:
activating a training mode of a robot;
steering the robot from a start location to a first target location in an environment to be disinfected;
recording in disinfection plan data at least one of commands applied to the robot to steer to the first target location and a measured position of the first target location;
recording in the disinfection plan data first timing information for operation of a UV disinfector on the robot at the target location; and
activating a disinfection mode of the robot in the environment, the disinfection mode causing the robot to use the disinfection plan data to navigate to the first target and to operate the UV disinfector according to the first timing information in the disinfection plan data.

20. The method of claim 19, further comprising:
steering the robot from the first target location to a second target location in the environment to be disinfected;
recording in the disinfection plan data at least one of commands applied to the robot to steer to the second target location and a measured position of the second target location; and
recording in the disinfection plan data second timing information for operation of a UV disinfector on the robot at the target location, wherein
activating the disinfection mode of the robot, further causes the robot to use the disinfection plan data to navigate to the second target and to operate the UV disinfector according to the second timing information in the disinfection plan data.
